(19) United States
(12) Patent Application Publication
(10) Pub. No.: US 2025/0238926 A1
(43) Pub. Date: Jul. 24, 2025

(54) ARTIFICIAL INTELLIGENCE ENABLED SUB-CLASSIFICATIONS OF DISEASE STATES

(71) Applicant: Digital Diagnostics Inc., Coralville, IA (US)

(72) Inventor: Michael D. Abramoff, University Heights, IA (US)

(21) Appl. No.: 19/033,378

(22) Filed: Jan. 21, 2025

Related U.S. Application Data
(60) Provisional application No. 63/623,698, filed on Jan. 22, 2024.

(51) Int. Cl. G06T 7/00; G16H 30/40

(52) U.S. Cl. CPC G06T 7/0012; G16H 30/40; G06T 2207/20081

(57) ABSTRACT

A fully autonomous system is used to subclassify a disease in a patient. For example, a tool receives one or more images of a body part of a patient, inputs the one or more images into a diagnostic model, and receives, as output from the diagnostic model, a diagnosis for the patient. The tool determines whether the diagnosis is positive for a given disease, and, responsive to determining that the diagnosis is positive, inputs a representation of the one or more images into a diagnosis subclassification model. The tool determines, based on output from the diagnosis subclassification model, a subclassification for the diagnosis, and outputs a control signal based on the subclassification.

### CROSS REFERENCE TO RELATED APPLICATIONS

This application claims the benefit of U.S. Provisional Patent Application Ser. No. 63/623,698, filed Jan. 22, 2024, which is incorporated by reference herein in its entirety.

### BACKGROUND

Current medical regulatory and legal frameworks guide processes and systems for classifying diagnoses, symptoms, and procedures. A common framework, known as the International Classification of Diseases, Tenth Revision (ICD-10), establishes a rubric through which physicians and other providers may classify these medical diagnoses and procedures, using a categorical nomenclature. If one is to instead use an autonomous artificial intelligence having outputs that do not map to ICD-10, one may need to go through immense regulatory hurdles to be approved for doing so, given that without mapping to ICD-10, new studies may need to be conducted that involve clinical trials requiring huge expense and participation. For example, if one is to use a fully-autonomous artificial diagnosis system that diagnoses in a dichotomous manner, i.e. “severity less than” vs “severity at least”, because those are the clinically relevant output categories that lead to appropriate management and treatment, these almost never map to ICD-10 codes as that is a categorical classification system. Thus, being able to both diagnose clinically relevant disease categories (including absence of disease) and being able to map the output to ICD-10 is not addressed under current solutions.

### SUMMARY

To address the aforementioned challenges, systems and methods are proposed herein following a new conceptualization, development and validation of an AI system specifically tuned towards hierarchical coding (e.g., a sub-classification system, such as ICD-10), that then will have outputs that map to the hierarchical coding-these are clinically less or even not relevant at all. Two diagnostic AI systems may be created: one (a diagnostic model) for the clinical diagnosis to improve clinical outcomes, health equity, improve access etc., and the other (the sub-classification model) solely to match the ICD-10 system (or other hierarchical framework).

Diabetic Macular Edema (DME) in the context of diabetic retinopathy or Diabetic Retina Disease (DRD) is one example of such a dis-mapping, where ICD-10 requires both DRD and DME to be evaluated, and furthermore, while ICD-10 allows for a diagnosis of DME where a diagnosis of DRD has previously been made, it does not allow DME to be diagnosed without the presence of DRD, even though such patients have relatively high prevalence. Having built a clinically relevant AI to diagnose those cases that have DME or DRD (both requiring similar management) from those that have neither, such output (DME OR DRD) cannot map to any ICD-10 code. The present disclosure addresses this issue by instructing a model to perform a secondary separate diagnosis of DME after a diagnosis of DRD is made, without going through the process of creating an entirely new AI system that diagnoses the various ICD-10 categories for DRD and DME (but would not be clinically very relevant, and would require much larger N for confirmatory studies to satisfy the aforementioned regulatory hurdles).

Systems and methods are described herein for sub-classifying a diagnosis using artificial intelligence in compliance with the regulatory framework. That is, where a study has already been done that resulted in a clinically useful diagnostic system, for example outputting a higher level ICD-10 code, a dependent sub-classification (e.g., having a deeper level ICD-10 code) may be determined by a novel AI subcomponent (e.g., a subclassification model) whose output can subclassify the output of the main AI, without requiring the building of a new AI, only for the purpose of outputting the finer grained ICD-10 code without clinical utility. Such a sub-classification model employs an artificial intelligence sub-classification within a hierarchical framework without requiring the rigor of a new study. To this end, the system first determines one or more independent diagnoses for a patient. Where at least one of the diagnoses matches a framework code (e.g., an ICD-10 code), the system additional AI goes on to generate a sub-classification for the disease that follows the framework codes using machine learning.

In some embodiments, a subclassification tool receives one or more images of a body part of a patient. The subclassification tool inputs the one or more images into a diagnostic model, and receives, as output from the diagnostic model, a diagnosis for the patient. The subclassification tool determines whether the diagnosis is positive for a given disease, and, responsive to determining that the diagnosis is positive, the subclassification tool inputs a representation of the one or more images into a diagnosis subclassification model. The subclassification tool determines, based on output from the diagnosis subclassification model, a subclassification for the diagnosis and outputs a control signal based on the subclassification.

### BRIEF DESCRIPTION OF THE DRAWINGS

FIG. 1 is an illustrative block diagram of components used in a system for applying sub-classifications in accordance with a framework, in accordance with an embodiment.

FIG. 2 is an illustrative diagram of modules of a sub-classification tool used to produce diagnosis sub-classifications in accordance with a framework.

FIG. 3 is an illustrative diagram of a data flow for generating sub-classifications, in accordance with an embodiment.

FIG. 4 is an illustrative diagram of a two-stage diagnostic model used to autonomously generate a probability-based diagnosis of a disease.

FIG. 5 is an illustrative diagram of a two-stage subclassification model used to autonomously generate one or more subclassifications of a disease.

FIG. 6 is an illustrative flowchart of a process for determining a sub-classification of a diagnosis, in accordance with an embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Overview

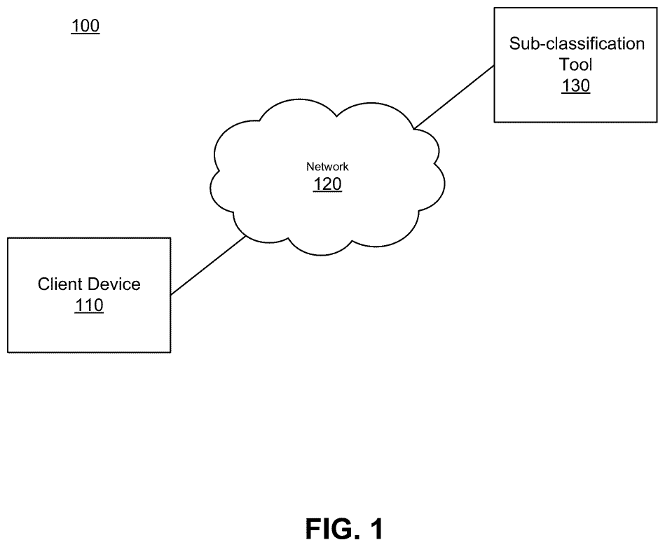

FIG. 1 is an illustrative block diagram of components used in a system for applying sub-classifications in accordance with a framework, in accordance with an embodiment. As depicted in FIG. 1, environment 100 includes client device 110, network 120, and sub-classification tool 130. Client device 110 may be any device or collection of devices used to capture signals from a patient. A primary example of client device 110 may be an imaging device used to capture images of a body part of a patient. The images may be of an exterior of the body, of an interior of the body (e.g., ophthalmic imaging such as OCT, x-ray imaging, ultrasound imaging, and the like), or a combination thereof. Other examples of client device 110 may be other sensors that collect patient biometric information and/or repositories of health information about the patient. Client device 110 may also have a user interface for instructing one or more diagnoses be performed based on the signals captured from the patient and for outputting any resulting diagnosis.

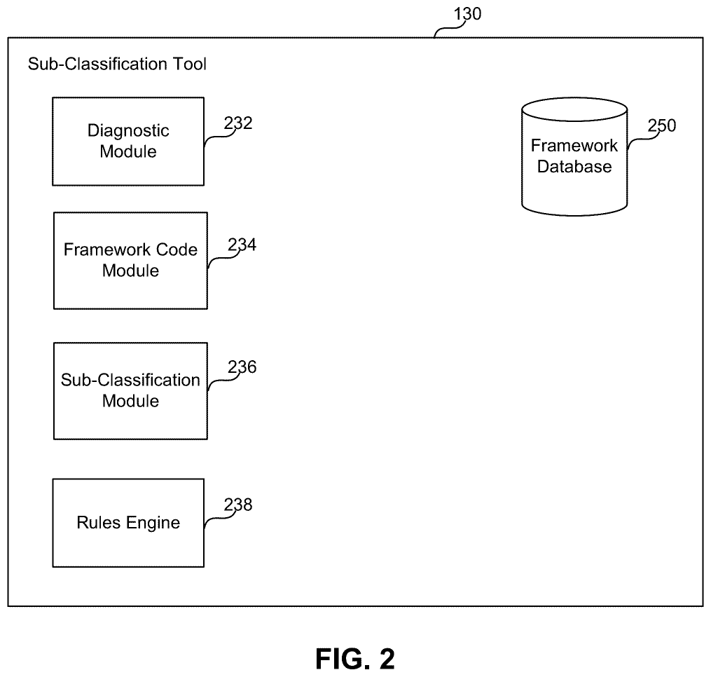

Network 120 may be any network, whether a local network (e.g., Wi-Fi, short-range link, mesh network, etc.) or global network (e.g., the Internet) that enables client device 110 and sub-classification tool 130 to communicate. Sub-classification tool 130 may output one or more diagnoses, including a sub-classification of a diagnosis where compatible with a framework. Further details of the sub-classification tool are described with respect to FIGS. 2-5 below.

FIG. 2 is an illustrative diagram of modules of a sub-classification tool used to produce diagnosis sub-classifications in accordance with a framework. As depicted in FIG. 2, sub-classification tool 130 includes diagnostic module 232, framework code module 234, sub-classification module 236, and rules engine 238, as well as framework database 250. The modules and databases depicted in FIG. 2 are merely exemplary, and more or fewer modules and/or databases may be used to achieve the functionality of sub-classification tool 130 disclosed herein.

Diagnostic module 232 autonomously generates a diagnosis of a disease using machine learning. To generate the diagnosis, the diagnostic module 232 receives one or more signals from client device 110, inputs those signals into one or more trained machine learning models, and determines a diagnosis based on the output of the one or more trained machine learning models. In an embodiment, one machine learning model is used, where historical training data of signals as labeled with disease diagnoses is used to train the machine learning model to make predictions. For example, retinal images may form the training set as labeled by whether the patient does or does not have diabetic retinopathy. In such single-model embodiments, however, there are downside risks of bias and lack of explainability. For example, where raw images are used to diagnose disease, factors wholly unrelated to the disease might be considered by the machine learning model, such as skin pigment. This can result in racial bias, where the machine learning model may inadvertently be trained to correlate disease with certain skin tones. This exacerbates explainability constraints, in that it is difficult to determine whether diagnoses are due to detection of disease or other factors (e.g., skin pigment).

In some embodiments, to avoid bias and explainability issues, two or more machine learning models are used. First, an extraction module may be used, where an image is input into the extraction module and the extraction module outputs biomarkers (e.g., artifacts within the eye, optionally paired with their locations within the eye). The biomarkers may then be input into a diagnosis model, trained using biomarkers extracted from training images and labeled with any diseases corresponding to the training images, where the diagnosis model may output probabilities of given diagnoses, from which one or more diagnoses may be determined. By abstracting away from the raw image, the possibility of bias creeping into the autonomous diagnosis is eliminated, and explainability is improved as it is only biomarkers that are considered by the model in determining whether the image is indicative of disease. Moreover, the subclassification tool does not backpropagate through both the extraction model and the diagnosis model, and the models are not otherwise trained together such that one is affected by the results of the other, which results in the transparency and explainability and further reduces bias. Exemplary two-stage diagnostic models are discussed in commonly-owned U.S. Pat. No. 11,790,523, entitled “Autonomous Diagnosis of a Disorder in a Patient from Image Analysis,” filed Oct. 30, 2018, issued Oct. 17, 2023, the disclosure of which is hereby incorporated by reference herein in its entirety. Further discussion in the context of diagnosis of ear diseases of two-stage models is disclosed in commonly-owned U.S. Pat. No. 11,786,148, entitled “Autonomous Diagnosis of Ear Diseases from Biomarker Data,” filed Aug. 1, 2019, issued Oct. 17, 2023, the disclosure of which is hereby incorporated by reference herein in its entirety. Discussion of obtaining images for autonomous diagnosis is disclosed in the aforementioned patents, and is also discussed in commonly-owned U.S. Pat. No. 11,232,548, entitled “System and Methods for Qualifying Medical Images, filed March 22, 201, granted Jan. 25, 2022, the disclosure of which is hereby incorporated by reference herein in its entirety.

In some embodiments, diagnostic module 232 may perform diagnostics using a vision transformer. The image may be tiled in high resolution, and the tiles may be transformed into embeddings and input into a vision transformer. The vision transformer may directly output a diagnosis and/or may output feature extractions from which a diagnostic model such as the diagnostic model mentioned in the foregoing may determine a diagnosis. Systems and methods of using a vision transformer for these purposes and others are hereby incorporated by reference herein in U.S. patent application Ser. No. 18/955,627, filed Nov. 21, 2024, entitled “High Resolution Medical Image Processing using Vision Transformers and Machine Learning,” the disclosure of which is hereby incorporated by reference herein in its entirety.

Framework code module 234 determines whether a given framework allows for a sub-classification of a disease. Framework database 250 may include a data structure of the framework. The framework may be in a tree format, where the tree indicates eligible sub-classifications that can be made based on a diagnosis. For example, given a framework of ICD-10, DME is not eligible to be diagnosed without DRD having first been diagnosed. Therefore, framework code module 234 may determine, responsive to a diagnosis, whether that diagnosis is reflected in framework database 250, and if so, whether there are eligible leaf nodes from that diagnosis of candidate sub-classifications that can be detected. Other exemplary frameworks may include other ICD-X codes (e.g., ICD-8, 9, and 11), Systematized Nomenclature of Medicine Clinical Terms (SNOMED), and the like. The frameworks are hierarchical, where only branches can be traversed, and one cannot jump from leaf to leaf. That is, a major problem the sub-classification model solves is caused by this one dimensional tree like-structure imposed by ICD-type frameworks, rather than a multi-dimensional/multi-axial classification space where the different categories are independent rather than fully dependent as is the case in ICD.

Sub-classification module 236 performs a sub-classification of a diagnosis where framework code module 234 determines that the diagnosis is eligible for sub-classification. Sub-classifications may have manifold purposes. They may be used to provide more specific or additional diagnoses of a patient condition, and may therefore be used to determine interventions to improve the patient's condition. Additionally or alternatively, the sub-classifications may be used to code a patient's condition for any purpose, such as for use in downstream processing of a patient visit such as filing a record in a patient's file, determining a bill code for the patient visit, and so on. Sub-classifications may be used as search parameters. For example, academic research on a particular condition may be performed by pulling public patient records having certain ICD-10 codes to form a data set. Moreover, reports may be generated based on ICD-10 codes. For example, to determine deaths resulting from COVID-19 or any other pandemic and differentiate them from other deaths occurring at that time, ICD-10 codes may be used to obscure deaths caused by other causes from those deaths directly resulting from COVID-19.

In some embodiments, outputs of hierarchical (e.g., ICD-10) codes may trigger user interface components. For example, sub-classification module 236 may generate a higher-order ICD-10 code (relative to orders within the hierarchy of the framework) and output the higher-order code along with options for selection for sub-refining to a lower-order code to a physician. Responsive to receiving a selection of a selectable option of a lower-order code, sub-classification module 236 may assign the selected code as the proper sub-classification. In some embodiments, sub-classification module 236 may output such selectable options responsive to detecting that a confidence level for candidate lower-order sub-classifications is below a minimum threshold.

Sub-classification module inputs a representation of the signals into a machine learning model (e.g., a subclassification model). For example, the images themselves and/or biomarkers extracted from the images may be input into the machine learning model by sub-classification module 236. In some embodiments, other auxiliary information may also be input into the machine learning model, such as intervention information (e.g., prescription, remediation recommended by a physician, etc.). The machine learning model may be trained to output sub-classifications based on a training set of images or representations thereof as labeled with sub-classifications (e.g., labeled with diagnoses). Subclassifications may be performed automatically responsive to determining that a subclassification may exist.

In some embodiments, diagnostic module 232 may have already determined probabilities of diagnoses that were not eligible for classification without precedent classification of a given diagnosis. In such embodiments, rather than input into a sub-classification model, sub-classification module 236 may instead go back to diagnoses determined using diagnostic module 232 and determine whether any of those diagnoses are sub-classifications under the framework of a given diagnosis, and may determine those diagnoses to be sub-classifications where they are indicated by the framework.

Rules engine 238 may take the sub-classifications and apply rules to the sub-classifications to determine downstream processing. The downstream processing may include filing away patient records using a certain code, processing information for a patient visit, and so on. Rules engine 238 may output one or more control signals determined on the basis of the applied rules.

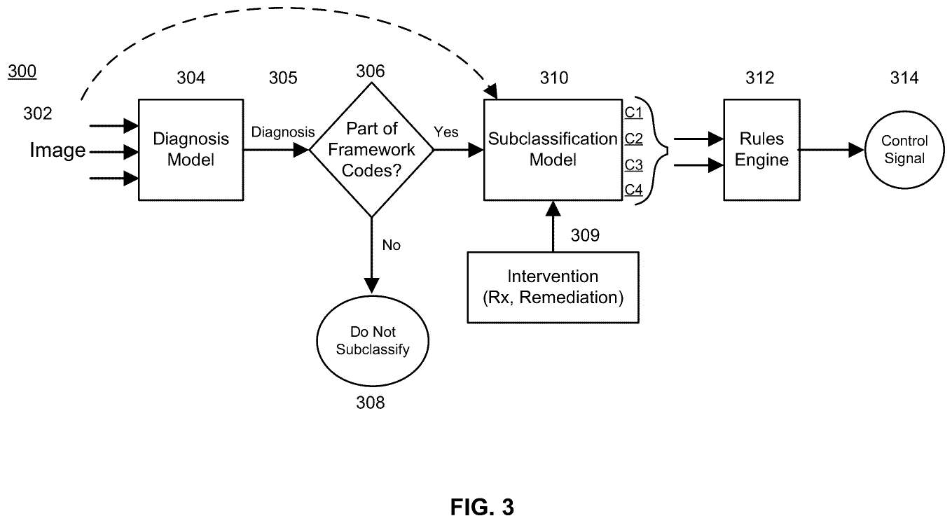

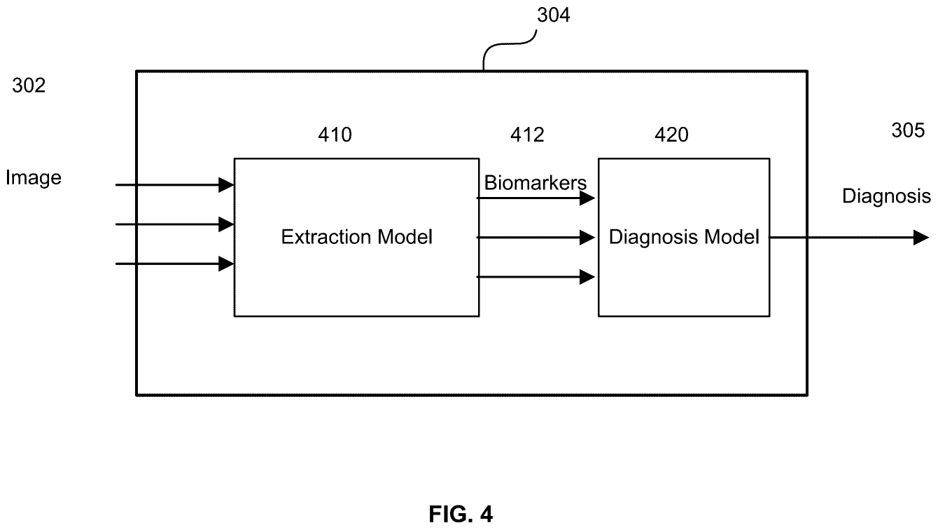

FIG. 3 is an illustrative diagram of a data flow for generating sub-classifications, in accordance with an embodiment. Data flow 300 begins with image 302 being input into diagnostic model 304 (e.g., by diagnostic module 232), image 302 being a signal as described in the foregoing and being exemplary, where other signals may be used additionally or alternatively. Diagnostic module outputs diagnosis 305. Turning briefly to FIG. 4 to provide more detail on diagnostic model 304 for embodiments where diagnostic model 304 is a two-stage model, FIG. 4 is an illustrative diagram of a two-stage diagnostic model used to autonomously generate a probability-based diagnosis of a disease. Image 302 is input into diagnostic model 304, which in some embodiments includes two separate supervised machine learning models, one being extraction model 410 and the other being diagnosis model 420. In some embodiments, image 302 includes a vision transformer, either as an extraction model 410 or a diagnosis model 420. Extraction model 410 (where used) outputs biomarkers 412 (e.g., artifacts within an image along with their locations within the image), which are then input into diagnosis model 420. Diagnosis model 420 outputs diagnosis 305 (e.g., by outputting probabilities of given candidate diagnoses, where diagnosis 305 includes one or more diagnoses having at least a threshold probability).

Returning to FIG. 3, sub-classification tool 130 determines 306 (e.g., using framework code module 234) whether the diagnosis is positive for a given disease that is specified within codes of a framework (e.g., does the framework have this disease coded into it?). Responsive to determining that the diagnosis is not referenced by the framework codes, sub-classification tool 130 determines 308 not to sub-classify the diagnosis. Responsive to determining that the diagnosis is referenced by the framework codes, sub-classification tool 130 provides a representation of image 302 (e.g., the image itself and/or biomarkers 412) to sub-classification model 310. Sub-classification model 310 may take auxiliary input 309 as input as well (e.g., intervention information including prescription or other remediation information). Sub-classification model 310 may determine probabilities that candidate sub-classifications apply, and those probabilities may be input to rules engine 312. Different sub-classification models may be used for different diagnoses, each one tuned to determining sub-classifications of their respective diagnosis using training data only for sub-classifications of those diagnoses. Alternatively, one sub-classification model may be used for all sub-classifications. Sub-classification model 310 may directly output sub-classifications (e.g., for those that have probabilities that exceed a threshold). Alternatively, rules engine 312 may determine sub-classifications by applying rules to the probabilities.

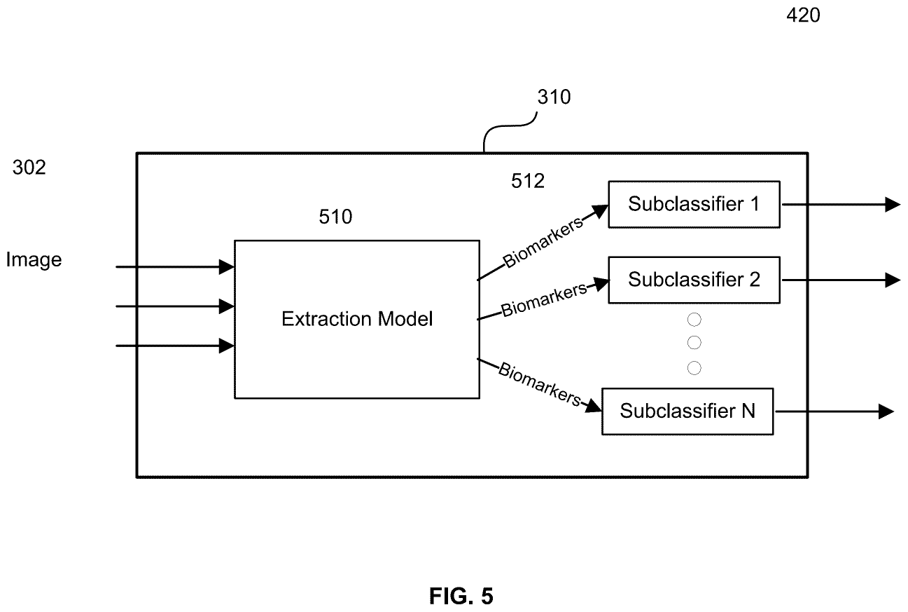

To more clearly describe the embodiment where the image 302 itself is input into subclassification model 310, we briefly turn to FIG. 5. FIG. 5 is an illustrative diagram of a two-stage subclassification model used to autonomously generate one or more subclassifications of a disease. As depicted in FIG. 5, image 302 (e.g., among other signals) is input into extraction model 510. Extraction model 510 may operate in the same manner as extraction model 410, resulting in biomarkers 512. Biomarkers 512 may be input into a same subclassifier in an embodiment, the single subclassifier outputting probabilities for various subclassifications. However, as depicted, subclassification model 310 may be a multi-task model, having a shared layer as an extraction model 510, and having siloed branches 512 for subclassifiers that each are trained to determine a different subclassification. The layers of the different branches of sub-classifiers 1-N are not backpropagated to extraction model 510 or to other branches, thereby ensuring maximal explainability and minimal bias from features that are encountered by extraction model 510 in image 302. Each sub-classifier outputs its probability for its respective subclassification that it is trained to predict, which is then output for further processing by rules engine 312.

Returning to FIG. 3, rules engine 312 may determine, using logic for each sub-classification, a next step to take. Rules engine 312 may determine which sub-classifications to apply based on the probabilities. For example, different threshold minimum probabilities may need to be present to conclude different sub-classifications, some having a higher threshold minimum than others. This may include applying a code for one or more of the sub-classifications based on their probabilities, forwarding a record to a storage location, adding a record to a report compendium, and so on.

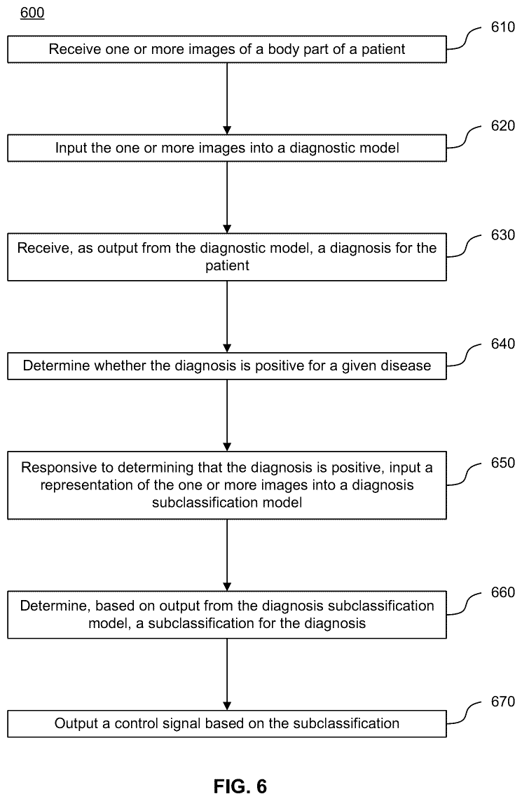

FIG. 6 is an illustrative flowchart of a process for determining a sub-classification of a diagnosis, in accordance with an embodiment. Process 600 may be executed by one or more processors executing instructions that cause modules of sub-classification tool 130 to perform the operations of the process. Process 600 begins with sub-classification tool 130 receiving 610 one or more images of a body part of a patient (e.g., image 302). Sub-classification tool 130 inputs 620 the one or more images into a diagnostic model, and receives 630, as output from the diagnostic model, a diagnosis for the patient (e.g., using diagnostic module 232).

Sub-classification tool 130 determines 640 whether the diagnosis is positive for a given disease (e.g., a disease indicated by framework 250, as determined using framework code module 234). Responsive to determining that the diagnosis is positive, sub-classification tool 130 inputs 650 a representation of the one or more images into a diagnosis subclassification model (e.g., using sub-classification module 236). Sub-classification tool 130 determines 660, based on an output from the diagnosis sub-classification model, a sub-classification for the diagnosis and outputs 670 a control signal based on the sub-classification (e.g., using rules engine 238).

### Summary

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for sub-classifying a diagnosis, the method comprising:
receiving one or more images of a body part of a patient;
inputting the one or more images into a diagnostic model;
receiving, as output from the diagnostic model, a diagnosis for the patient;
determining whether the diagnosis is positive for a given disease;
responsive to determining that the diagnosis is positive, inputting a representation of the one or more images into a diagnosis subclassification model;
determining, based on output from the diagnosis subclassification model, a subclassification for the diagnosis; and
outputting a control signal based on the subclassification.

2. The method of claim 1, wherein the diagnostic model comprises an extraction model and a diagnosis model and produces the diagnosis by:
inputting the one or more images into the extraction model;
receiving, as output from the extraction model, biomarkers indicative of disease;
inputting the biomarkers into the diagnosis model; and
receiving indicia of the diagnosis from the diagnosis model.

3. The method of claim 2, wherein determining whether the diagnosis is positive for a given disease comprises determining whether the diagnosis matches one of a plurality of predefined diseases for which subclassification is eligible.

4. The method of claim 1, wherein the representation of the one or more images that is input into the diagnosis subclassification model comprises biomarkers extracted from the one or more images by an extraction model.

5. The method of claim 1, wherein the diagnosis subclassification model is trained using a training set comprising historical representations of medical images as labeled with one or more subclassifications.

6. The method of claim 1, wherein determining the subclassification comprises inputting the output from the diagnosis subclassification model into a rules engine, the rules engine determining the subclassification.

7. The method of claim 1, wherein the control signal is determined from a plurality of candidate control signals based on the subclassification.

8. A computer program product for sub-classifying a diagnosis, the computer program product comprising a computer-readable storage medium containing computer program code for:
receiving one or more images of a body part of a patient;
inputting the one or more images into a diagnostic model;
receiving, as output from the diagnostic model, a diagnosis for the patient;
determining whether the diagnosis is positive for a given disease;
responsive to determining that the diagnosis is positive, inputting a representation of the one or more images into a diagnosis subclassification model;
determining, based on output from the diagnosis subclassification model, a subclassification for the diagnosis; and
outputting a control signal based on the subclassification.

9. The computer program product of claim 8, wherein the diagnostic model comprises an extraction model and a diagnosis model and produces the diagnosis by:
inputting the one or more images into the extraction model;
receiving, as output from the extraction model, biomarkers indicative of disease;
inputting the biomarkers into the diagnosis model; and
receiving indicia of the diagnosis from the diagnosis model.

10. The computer program product of claim 9, wherein determining whether the diagnosis is positive for a given disease comprises determining whether the diagnosis matches one of a plurality of predefined diseases for which subclassification is eligible.

11. The computer program product of claim 8, wherein the representation of the one or more images that is input into the diagnosis subclassification model comprises biomarkers extracted from the one or more images by an extraction model.

12. The computer program product of claim 8, wherein the diagnosis subclassification model is trained using a training set comprising historical representations of medical images as labeled with one or more subclassifications.

13. The computer program product of claim 8, wherein determining the subclassification comprises inputting the output from the diagnosis subclassification model into a rules engine, the rules engine determining the subclassification.

14. The computer program product of claim 8, wherein the control signal is determined from a plurality of candidate control signals based on the subclassification.

15. A system comprising:
memory with instructions for sub-classifying a diagnosis encoded thereon; and
one or more processors that, when executing the instructions, are caused to perform operations comprising:
receiving one or more images of a body part of a patient;
inputting the one or more images into a diagnostic model;
receiving, as output from the diagnostic model, a diagnosis for the patient;
determining whether the diagnosis is positive for a given disease;
responsive to determining that the diagnosis is positive, inputting a representation of the one or more images into a diagnosis subclassification model;
determining, based on output from the diagnosis subclassification model, a subclassification for the diagnosis; and
outputting a control signal based on the subclassification.

16. The system of claim 15, wherein the diagnostic model comprises an extraction model and a diagnosis model and produces the diagnosis by:
inputting the one or more images into the extraction model;
receiving, as output from the extraction model, biomarkers indicative of disease;
inputting the biomarkers into the diagnosis model; and
receiving indicia of the diagnosis from the diagnosis model.

17. The system of claim 16, wherein determining whether the diagnosis is positive for a given disease comprises determining whether the diagnosis matches one of a plurality of predefined diseases for which subclassification is eligible.

18. The system of claim 15, wherein the representation of the one or more images that is input into the diagnosis subclassification model comprises biomarkers extracted from the one or more images by an extraction model.

19. The system of claim 15, wherein the diagnosis subclassification model is trained using a training set comprising historical representations of medical images as labeled with one or more subclassifications.

20. The system of claim 15, wherein determining the subclassification comprises inputting the output from the diagnosis subclassification model into a rules engine, the rules engine determining the subclassification.
